(19) United States
(12) Patent Application Publication
(10) Pub. No.: US 2020/0110054 A1
(43) Pub. Date: Apr. 9, 2020

(54) MICROFLUIDIC DEVICES AND METHODS USING THE SAME

(71) Applicant: TECHNION RESEARCH & DEVELOPMENT FOUNDATION LIMITED, Haifa (IL)

(72) Inventors: Moran BERCOVICI, Haifa (IL); Marianna TRUMAN, Haifa (IL)

(21) Appl. No.: 16/499,619

(22) PCT Filed: Apr. 3, 2018
(86) PCT No.: PCT/IL2018/050391

§ 371 Date: Sep. 30, 2019

Related U.S. Application Data
(60) Provisional application No. 62/480,571, filed on Apr. 3, 2017.

(51) Int. Cl. G01N 27/447; G01N 1/28; B01L 3/00; C12Q 1/04

(52) U.S. Cl. CPC G01N 27/44791; G01N 27/44704; B01L 2300/0681; B01L 3/502753; C12Q 1/04; G01N 1/28

(57) ABSTRACT

An isotachophoresis (ITP) apparatus having a first zone configured to contain a solution comprised of a trailing electrolyte (TE); a second zone configured to contain solution containing a leading electrolyte (LE); a flow channel connecting the first zone and the second zone; and a first filter having a pore size sufficient to entrap an analyte, the first filter being integrated within the first zone, and in fluid communication with the flow channel, wherein the flow channel is in a distinct direction with respect to the filtration flow, is disclosed herein. A system comprised of a microfluidic device comprised of a flow channel is further disclosed. A method of electrophoresis-sample preparation is further disclosed herein.

### CROSS REFERENCE TO RELATED APPLICATIONS

This application claims the benefit of priority from U.S. Provisional Patent Application No. 62/480,571, filed on Apr. 3, 2017. The content of the above document is incorporated by reference in its entirety as if fully set forth herein.

### FIELD OF THE INVENTION

The present invention, in some embodiments thereof, relates to methods of sample preparation for microfluidic applications where detection of particles of a given size range are desired.

### BACKGROUND OF THE INVENTION

For applications that require electromagnetic fields to be applied through the sample, such as electrophoresis, some biological samples present a challenge due to high salt concentration which results in a significant joule-heating as well as sub-optimal conditions for detection and reaction.

Moreover, some applications target detection of particles of a given size range (e.g. bacteria), while rejecting particles outside that size range (e.g. red blood cells and proteins on one end of the spectrum, and white blood cells on the other).

This is particularly relevant for urine, where for detection of urinary tract infections one is interested in bacterial content. One way to reduce the salt concentration in urine is to dilute the sample. However, this results in proportional lowering of bacteria concentration and thus in reduced detection capabilities.

If separation of biological species is desired, the current method of choice is conventional centrifugation. While it enables separation of the bacteria from the supernatant high salt liquid, centrifugation requires a large sample volume, is labor intensive, runs into difficulties when a well-defined particle size range is required and is generally not applicable to point of care situations.

### SUMMARY OF THE INVENTION

The present invention, in some embodiments thereof, relates to methods of sample preparation for microfluidic applications where detection of particles of a given size range are desired.

According to an aspect of some embodiments of the present invention there is provided an isotachophoresis (ITP) apparatus comprising: (i) a first zone configured to contain a solution comprising a trailing electrolyte (TE); (ii) a second zone configured to contain solution comprising a leading electrolyte (LE); (iii) a flow channel connecting the first zone and the second zone; and (iv) a first filter having a pore size sufficient to entrap an analyte, the first filter being integrated within the first zone, and in fluid communication with the flow channel; wherein the flow channel is in a distinct direction with respect to the filtration flow.

In some embodiments, the first filter and the flow channel are substantially in the same plane.

In some embodiments, the first filter has a pore size in the range of 0.1-1.0 μm.

In some embodiments, the apparatus further comprises a second filter having a pore size larger than the analyte.

In some embodiments, the second filter is in fluid communication with the first filter.

In some embodiments, the second filter is a detachable filter disposed atop the first filter.

In some embodiments, the second filter is characterized by a pore size in the range of 0.5-10 μm.

In some embodiments, the first zone and the second zone are configured to be operably connected to at least one anode and at least one cathode.

According to another aspect of some embodiments of the present invention there is provided a system comprising: (i) a microfluidic device comprising a flow channel; (ii) a first filter having a pore size sufficient to entrap an analyte, and in fluid communication with the flow channel, the flow channel is in a distinct direction with respect to the filtration flow; (iii) a receptacle divided by a membrane into a first compartment configured to contain a fluid sample and a second compartment configured to contain a buffer, wherein the first compartment of the receptacle is configured to be placed in fluid communication with the flow channel through the filter, the receptacle comprises a membrane-opening mechanism configured to allow flow of the buffer to the flow channel subsequent to flow of the fluid sample through the filter.

In some embodiments, the system further comprises further comprises a second filter having a pore size larger than the analyte.

In some embodiments, the second filter is in fluid communication with the first filter. In some embodiments, the second filter is a detachable filter disposed atop the first filter.

In some embodiments, the microfluidic device of the system is an ITP apparatus comprising: (i) a first zone configured to contain a solution comprising a TE; (ii) a second zone configured to contain solution comprising an LE; the flow channel connects the first zone and the second zone, and the first zone and the second zone are configured to be operably connected to at least one anode and at least one cathode.

According to another aspect of some embodiments of the present invention there is provided an electrophoresis-sample preparation method comprising: (i) a filtration step comprising filtering a fluid sample comprising an analyte through a first filter sufficient to entrap the analyte; (ii) and a buffer exchange step comprising passing an electrophoresis buffer through the first filter; thereby receiving an electrophoresis buffer comprising the analyte.

In some embodiments, the filtration step comprises a preliminary filtration step of filtering the fluid sample through a second filter having a pore size larger than the analyte.

In some embodiments, the method comprises a step of labeling the analyte with a label detected under electrophoresis.

In some embodiments, the method comprises step (iii) comprising: applying the electrophoresis buffer comprising the analyte to a flow channel, applying an electric potential along the flow channel, and detecting the analyte. In some embodiments, the electrophoresis is ITP and the electrophoresis buffer is a solution comprising a TE. In some embodiments, the fluid sample is urine.

In some embodiments, the analyte is bacteria. In some embodiments, the second filter is sufficient to remove white blood cells from the fluid sample.

In some embodiments, the method is for detection of urinary tract infections (UTI).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show non-limiting exemplary set up for the steps of a buffer exchange & filtering method disclosed herein. “1” denotes empty syringe; “2” denotes buffer syringe; “3” denotes filters; “4” denotes urine sample; “5” denotes bacteria; “6” denotes blood cells; “7” denotes buffer solution; “8” denotes switched valve; “9” denotes bacteria in buffer.

FIGS. 2A-C show schematic side-view illustrations of non-limiting configurations of ITP apparatus.

FIGS. 3A-F show exemplary non-limiting configurations of the disclosed system integrated into a microfluidic chip.

FIGS. 4A-B present experimental results demonstrating the efficiency of filtration technique using urine samples spiked with bacteria (FIG. 4A) and real urinary tract infection (UTI) samples (FIG. 4B). Serial dilutions were performed for each urine sample, and bacteria number, before (“2” columns) and after (“1” columns) filtration, was evaluated using drop plate count method.

FIGS. 5A-B present direct bacteria focusing from urine samples following filtration: Demonstration of detected bacteria from a 10³ and 10⁶ CFU/mL (CFU: colony forming unit) filtered urine samples (FIG. 5A); Control sample was composed of buffers only (no bacteria). Scale bar: 100 μm. ITP focusing for control (black dashed line), 10³ cfu/mL (purpil line) and 10⁶ cfu/mL (blue line) filtered urine samples was quantified by plotting the values of maximum intensity of the fluorescence signal as a function of time over a fixed region of interest, simulating the signal of a point-detector (FIG. 5B).

FIGS. 6A-E present direct bacteria focusing from 10⁸ cfu/mL filtered urine sample: Demonstration of detected bacteria in the wide region (FIG. 6A), chamber and narrow regions (FIG. 6B) and in the vicinity of the anode reservoir (FIG. 6C); Scale bar: 100 μm; ITP focusing for 10⁸ cfu/mL (bright line) filtered urine samples was quantified by plotting the values of maximum intensity of the fluorescence signal as a function of time over a fixed region of interest (FIG. 6D). The inset is enlarged and presented in FIG. 6E, dark line.

### DETAILED DESCRIPTION

The present invention relates to electrophoresis devices and systems, including but not limited to, isotachophoresis (ITP).

The invention further provides methods of sample preparation for microfluidic applications, and specifically electrokinetic applications where detection of particles of a given size range are desired. The methods and devices of the invention allow for simple and rapid buffer exchange such as removal of salt from a sample (e.g., urine), thereby preventing joule-heating under the electrophoresis assay.

According to one aspect, there is provided a method for preparing a sample for electrophoresis, the method comprising:
(i) a filtration step comprising filtering a fluid sample comprising an analyte through a first filter sufficient to entrap the analyte; and
(ii) a buffer exchange step comprising passing a buffer through the first filter; thereby receiving a buffer comprising the analyte.

In some embodiments, the filtration step comprises a preliminary filtration step of filtering the fluid sample through a second filter having a pore size larger than the analyte.

In some embodiments, the method and the apparatus described below allows to detect the analyte while the particles of interest (e.g., bacterial cells) remain intact, e.g., in a non-lysis form prior to their detection.

In some embodiments, by “larger” it is meant to refer to 5 to 20% larger. In some embodiments, by “larger” it is meant to refer to 5 to 50% larger.

In some embodiments, by “larger” it is meant to refer to 1%, 5%, 10%, 15%, 20%, 25%, 30%, 35%, 40%, 45%, or 50% larger, including any value and range therebetween. In some embodiments, by “larger” it is meant to refer to at least 1%, at least 5%, or at least 10% larger, including any value and range therebetween.

In some embodiments, the method comprises step (iii) comprising: applying the electrophoresis buffer comprising the analyte to a flow channel. In some embodiments, step (iii) further comprises applying an electric potential along the flow channel. In some embodiments, step (iii) further comprises detecting the analyte. In some embodiments, step (iii) further comprises quantification of the analyte.

In some embodiments, the buffer is an electrophoresis buffer.

In some embodiments, the electrophoresis is isotachophoresis (ITP). In some embodiments, the electrophoresis buffer is a solution comprising a trailing electrolyte (TE).

In some embodiments, the electrophoresis buffer is a solution comprising a leading electrolyte (LE).

As used herein, the term “fluid sample” refers to a material suspected of containing an analyte. The fluid sample can be used directly as obtained, for example, from any biological source.

The fluid sample can also be obtained from an organism and the relevant portion extracted or dissolved into a solution.

In some embodiments, the fluid sample is a biological sample. In some embodiments, the biological sample is obtained from a subject (e.g., a mammal, a human). In some embodiments, the fluid sample is urine.

In some embodiments, the fluid sample is a blood sample.

A non-limiting example for a filtration comprises the filtration of white blood cells from other components in the blood sample.

The blood sample may be filtered through a filter having a mean pore size equal to or less than 5 microns resulting in immobilized white blood cells within the filter. Another non-limiting example comprises filtration of bacterial cells from larger debris in a saliva sample.

The filter may be in the form of membrane.

In some embodiments, bacterial cells are filtered from other components in a urine sample.

In some embodiments, bacterial cells are first separated from blood cells (e.g., leukocytes and erythrocytes) through a filter having a mean pore size of 2 to 5 microns, resulting in a filtrate comprising bacterial cells and a urine (water and solubles). In some embodiments, bacterial cells are thereafter separated from the urine and solubles through another filter having a mean pore size equal to or less than 0.5 micron resulting in immobilized bacterial cells in the second filter.

In some embodiments, bacterial cells are retrieved from the second filter by buffer exchange.

Another non-limiting example for filtration includes, but is not limited to, separation of erythrocytes from a blood sample.

In a non-limiting example, erythrocytes are separated from white blood cells (e.g., leukocytes) through a first filter having a mean pore size of 9 to 10 microns resulting in a filtrate comprising erythrocytes and blood fluids (such as serum, or plasma).

In some embodiments, erythrocytes are then separated from the blood fluids and solubles through a second filter having a mean pore size equal to or less than 3 microns resulting in immobilized erythrocytes in the filter;

In some embodiments, erythrocytes are retrieved from the second filter by buffer exchange.

In some embodiments, filtered cells remain intact and are subsequently analyzed intact. In another embodiment, filtered cells are subsequently lysed and are then assayed.

Non-limiting example of lysate analysis includes, but is not limited to, quantitation of nucleic acids (e.g., DNA and RNA) and proteins.

The term “analyte” refers to a substance to be detected or assayed by the method of the present invention. Non-limiting exemplary analyte may include, but are not limited to proteins, peptides, nucleic acid segments, molecules, cells (e.g., bacterial cells), microorganisms and fragments and products thereof. In some embodiments, the analyte comprises a plurality of particles, or one or more molecules of interest.

In some embodiments, the method is for detection of urinary tract infections (UTI). In some embodiments, the first filter is sufficient to entrap bacteria (i.e., being the analyte) from the fluid sample. In some embodiments, the second filter is sufficient to remove blood cells e.g., white blood cells, from the fluid sample.

In some embodiments, the detection may be assisted or enhanced by imaging, such as using a fluorescence-based technique.

In some embodiments, the method comprises a step of labeling the analyte with a label. In some embodiments, prior to the filtration step, the analyte (e.g., bacteria) is labeled with a dye (e.g., SYTO9). In some embodiments, the label is selected from, without being limited thereto, is a dye or a fluorescent. In some embodiments, the label is detectable under electrophoresis, e.g., ITP.

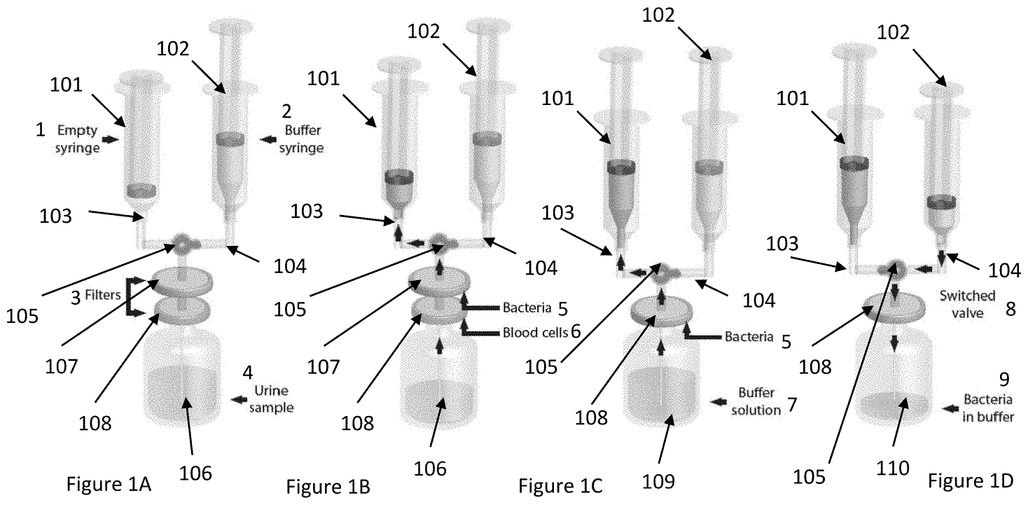

Reference is made to FIGS. 1A-D showing exemplary embodiments of a sample preparation method comprising a filtration step and a buffer exchange step. As a non-limiting example, the method may involve the usage of two sterile syringes 101 and 102, and two filter units 107 and 108 with respective pore sizes D1 and D2, where a characteristic size D of particles of interest (also referred to as analyte) is between D1 and D2.

Non-limiting characteristic values of D1 and D2 are in the range of 0.1-1.0 μm and 0.5-10.0 μm, respectively. Optionally, PVDF membranes from EMD Millipore may be used.

In some embodiments, such as for detection of UTI using a urine sample, filter 108 aims to exclude white blood cells and other debris larger than D2 from the final sample, while filter 107 aims to collect the bacteria by size exclusion for further analysis, while discarding items smaller than D1.

In some embodiments, the initial conditions include a vessel 106 comprising the fluid sample and the filtration device containing filters 107, 108, and syringes 101, 102. Optionally, syringe 101 is initially empty while the syringe 102 is filled with a desired buffer solution (e.g., a trailing electrolyte buffer for isotachophoresis).

In some embodiments, the filtration procedure comprises these steps:
(a) positioning valve 105 such that syringe 101 draws sample from the vial 106 through pipe 103;
(b) passing the sample from the vial 106 with syringe 101, through filters 108 and 107 and pipe 103, collecting the particles of interest (e.g., bacteria) with size D between D1 and D2 on the bottom side of filter 107, while leaving particles of size greater than D2 on the bottom side of the filter 108, and pulling particles of size less than D1 (the filtrate) into the syringe 101. Thus, at the end of this operation, syringe 101 contains buffer with small particles, while filter 108 has large debris. Filter 107 has the particles of interest, e.g. bacteria;
(c) removing filter 108;
(d) optionally washing filter 107 by replacing vial 106 with vial 109, containing the desired washing buffer, and pulling the buffer with syringe 101 in order to wash the particles of interest (e.g. bacteria) and to remove traces of interfering sample components;
(e) replacing buffer vial 109 with an empty vial 110, positioning valve 105 to allow flow from the buffer syringe 102 and elute the particles of interest from filter 107 to an empty vial 110.

Optionally, pushing the fluid is executed rather than pulling, where the following step may take place: (a) syringe 101 contains sample, syringe 102 contains buffer and the sample is pushed through filters D2 and D1 (arranged in this order), (b) filter D2 is removed, the valve positioned so to permit flow from syringe 102 through filter D1, the buffer from the buffer syringe 102 is passed through filter D1, to wash bacteria on filter D1 from traces of sample components (optional step), and (c) the bacteria is eluted from filter D1 into the buffer syringe 102.

As presented herein below, the performance of the provided method and the filtration system efficiency was evaluated in terms of bacteria losses, which may mainly occur due to possible binding to the filter membrane.

In a further embodiment, the filtration method is integrated in the microfluidic chip, thus enabling a single step operation.

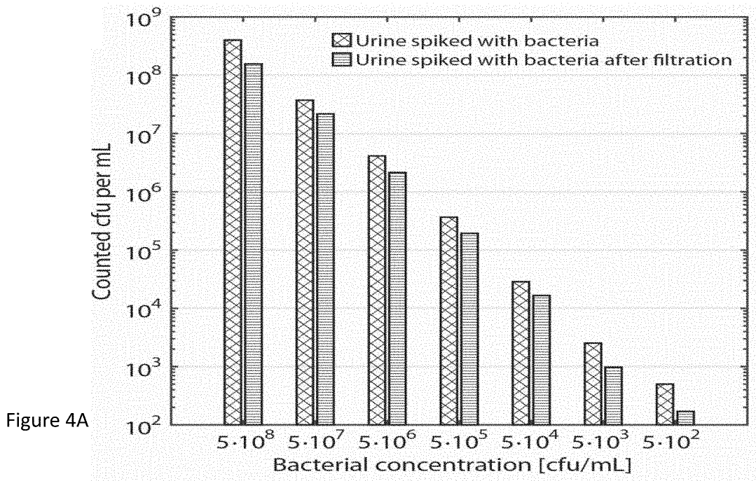

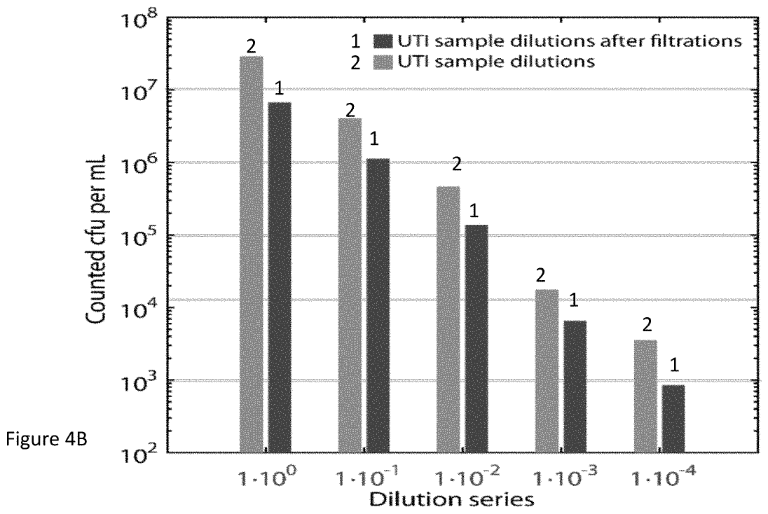

Reference is made to FIG. 4A showing experimental results comparing the number of retrieved bacteria processed under the described method, to the number of bacteria originally in the sample. Bacteria losses are mainly due to possible binding to the filter membrane, and can be reduced by pre-treatment, coatings and/or material optimization. The bacteria number yield of the embodiment shown is about 50% on average for urine samples spiked with bacteria, which is sufficient for many practical purposes such as subsequent UTI detection.

The Apparatus

According to another aspect, there is provided an isotachophoresis (ITP) apparatus comprising:
(i) a first zone configured to contain a first solution;
(ii) a second zone configured to contain a second solution;
(iii) a flow channel connecting the first zone and the second zone; and
(iv) a first filter having a pore size sufficient to entrap an analyte, the first filter being integrated within the first zone, and in fluid communication with the flow channel; wherein the flow channel is in a distinct direction with respect to the filtration flow.

In some embodiments, the first solution comprises a trailing electrolyte (TE). In some embodiments, the first solution comprises a leading electrolyte (LE). In some embodiments, the first solution comprises a leading electrolyte (LE). In some embodiments, the first solution comprises a trailing electrolyte (TE).

As used hereinthroughout, the term “fluid communication” means fluidically interconnected, and refers to the existence of a continuous coherent flow path from one of the components of the system to the other if there is, or can be established, liquid and/or gas flow through and between the ports, when desired, to impede fluid flow therebetween.

Optionally, by “sufficient to entrap” it is meant that upon contact with first filter 220, and upon an operation of a filtration flow, at least 10%, at least 20%, at least 30%, at least 40%, at least 50%, at least 60%, at least 70%, at least 80%, least 90%, at least 99%, or even approximately 100%, of the analyte remains within first the filter, e.g., the first filter 220 as described below.

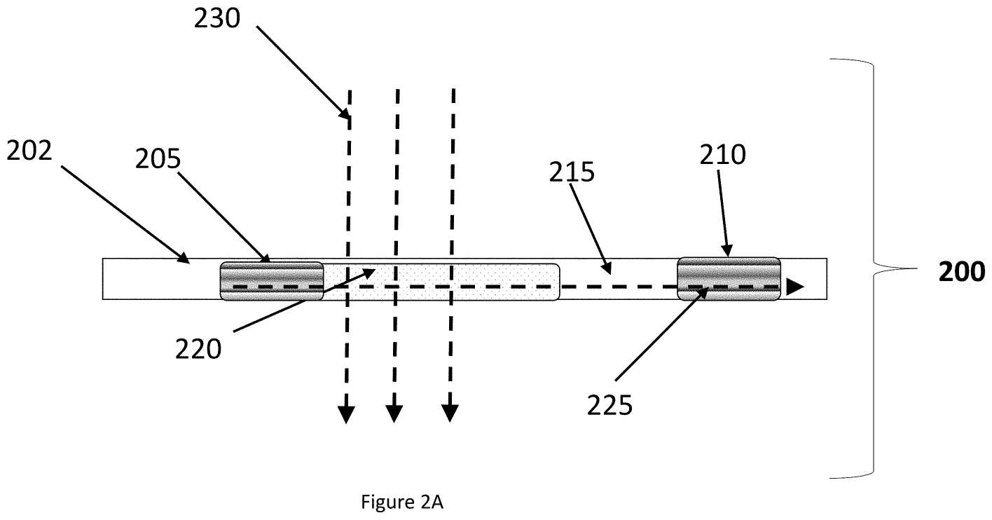

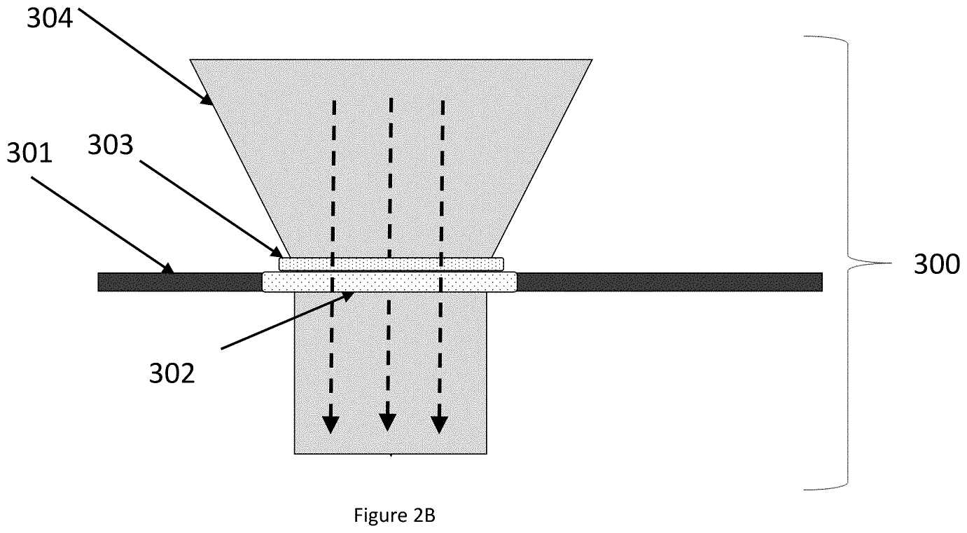

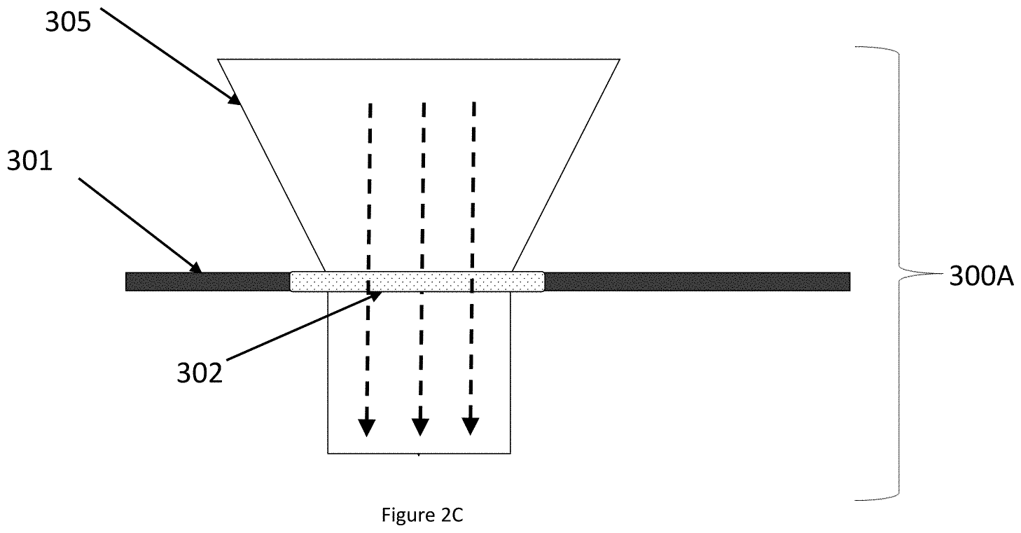

Reference is made to FIG. 2A showing an ITP apparatus 200 according to certain exemplary embodiments of the disclosed subject matter.

ITP Apparatus 200 has a microfluidic chip 202. Apparatus 200 may have a first zone 205. The first zone is configured to contain a first solution, e.g., a solution comprising a TE. Apparatus 200 may have a second zone 210. Second zone is configured to contain a second solution, e.g., a solution comprising an LE. ITP Apparatus 200 has a flow channel 215 connecting the first zone 205 and the second zone 210. ITP Apparatus 200 may have a first filter 220. First filter may be integrated within, or embedded to, first zone 205. The first filter 220 has a pore size sufficient to entrap an analyte.

Flow channel 215 may allow a flow direction 225 of the analyte e.g., within flow channel 215 upon operation of ITP apparatus 200 (when an electric potential is applied along the flow channel). The first filter 220 allows a filtering flow 230 of a sample.

Flow direction 225 and filtering flow 230 may be distinct.

By “distinct” or “distinct direction” it is meant that an axis of a flow of a liquid sample (i.e. filtering flow 230) passing through the first filter 220 is different from flow direction 225, such as by e.g., at least 10°, at least 20°, at least 30°, at least 40°, at least 50°, at least 60°, at least 70°, at least 80°, or at least 90°. Optionally, filtration flow 230 is substantially perpendicular to the longitudinally plane of flow channel 215.

Optionally, first filter 220 and flow channel 215 are substantially in the same plane. Optionally, first filter 220 is disposed within an internal space of flow channel 215.

Optionally, first filter 220 and flow channel 215 are adjacently and horizontally disposed to be side-by-side.

Optionally, first filter 220 has a pore size in the range of 0.1 to 5.0 μm, or optionally in the range of 0.1 to 1.0 μm. Optionally, first filter 220 has a pore size of 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μm, including any value and range therebetween.

Optionally first zone 205 and second zone 210 are configured to be operably connected to at least one anode or at least one cathode.

The term “operably connected to” means that the elements are connected either directly or indirectly. In a non-limiting example, “operably connected to” may refer to the capability of an anode or a cathode to directly or indirectly transfer an electric current to, or receiving an electric current from, first zone 205 and/or second zone 210.

Reference is made to FIG. 2B showing further exemplary embodiments of the disclosed ITP apparatus. ITP apparatus 300 may have a second filter 303. Optionally, second filter 303 may have a pore size larger than the analyte. Optionally, second filter 303 has a pore size in the range of 0.5 to 20 μm, or optionally in the range of 0.5 to 10 μm. Optionally, second filter 303 has a pore size of 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 μm, including any value and range therebetween. Optionally, second filter 303 is in fluid communication with first filter 302. Optionally, second filter 303 is a detachable filter disposed atop the first filter.

In a non-limiting exemplary configuration second filter 303 has a pore size of 1 to 6 μm, e.g., 1, 2, 3, 4, 5, or 6 μm, including any value and range therebetween, and first filter 302 has a pore size of less than 1 or less than 0.5 μm.

In another non-limiting exemplary configuration second filter 303 has a pore size of 7 to 12 μm, e.g., 7, 8, 9, 10, 11, or 12 μm, including any value and range therebetween, and first filter 302 has a pore size of less than 5 less than 4 less than 3 or less than 2 μm.

The term “atop” as used herein is not restricted to a particular orientation with respect to the gravitational field of the local environment, but simply refers to one element being disposed on another element, optionally with one or more intermediate elements disposed therebetween, unless otherwise indicated. Thus, a first element may be “atop” a second element even if the first element is disposed on a “bottom” (from the standpoint of gravity) surface of the second element.

The term “detachable” as used herein refers to members which can be easily removed, while maintaining the overall structure of the other members. Optionally, no tools such as screw drivers are needed for the detachment. Optionally, no excessive forces are need for the detachment.

ITP apparatus 300 may have a funnel 304, allowing a liquid sample to pass therethrough or to be pushed and to flow into first and second filters (302 and 303, respectively). Non-limiting embodiments of first and second filters are described in ITP Apparatus 200.

In exemplary configurations, second filter 303 may be part of the funnel 304. Further configurations of second filter 303 may be similar to the ITP Apparatus 200.

ITP apparatus 300 may have a microfluidic chip 301, and may have first zone, second zone, flow channel, filtering flow, all of which may be configured similarly to the ITP Apparatus 200.

In a non-limiting operation of apparatus 300, particles of interest (e.g. bacteria) are collected on the filter 302. Filter 303 may then be discarded.

FIG. 2C shows another optional configuration of ITP apparatus 300 (denoted as “300A”) following a step of replacing the funnel 304 by funnel 305, containing buffer (e.g., TE buffer). The buffer is then further pushed through filter 302, removing traces of the sample, and leaving the particles of interest (e.g. bacteria) clean and immersed in a well-defined buffer, ready to be processed by the microfluidic chip.

The System

According to another aspect there is provided a system comprising:
(i) a microfluidic device comprising a flow channel;
(ii) a first filter having a pore size sufficient to entrap an analyte, and in fluid communication with the flow channel, the flow channel is in a distinct direction with respect to the filtration flow;
(iii) a receptacle divided by a barrier into a first compartment configured to contain a fluid sample and a second compartment configured to contain a buffer.

Optionally, the first compartment of the receptacle is configured to be placed in fluid communication with the flow channel through the filter.

Optionally, the receptacle comprises a barrier-opening mechanism configured to allow flow of the buffer to the flow channel subsequent to flow of the fluid sample through the filter.

Optionally, the system has a second filter having a pore size larger than the analyte.

Optionally, second filter is in fluid communication with the first filter.

Optionally, second filter is a detachable filter disposed atop the first filter.

Optionally, the microfluidic device is an ITP microfluidic device comprising a first zone and a second zone.

Optionally, the first zone is configured to contain a solution comprising a trailing electrolyte (TE) and the second zone is configured to contain solution comprising a leading electrolyte (LE);

Optionally, the flow channel connects the first zone and the second zone. Optionally, the first zone and the second zone are configured to be operably connected to at least one anode and at least one cathode.

Non-limiting exemplary embodiments of the flow channel, the first filter, the second filter, the pore size, the analyte, and the distinct direction are described hereinabove under “the apparatus”.

Optionally, the barrier is in the form of a membrane.

Optionally, the barrier is deposited substantially parallel to the flow channel.

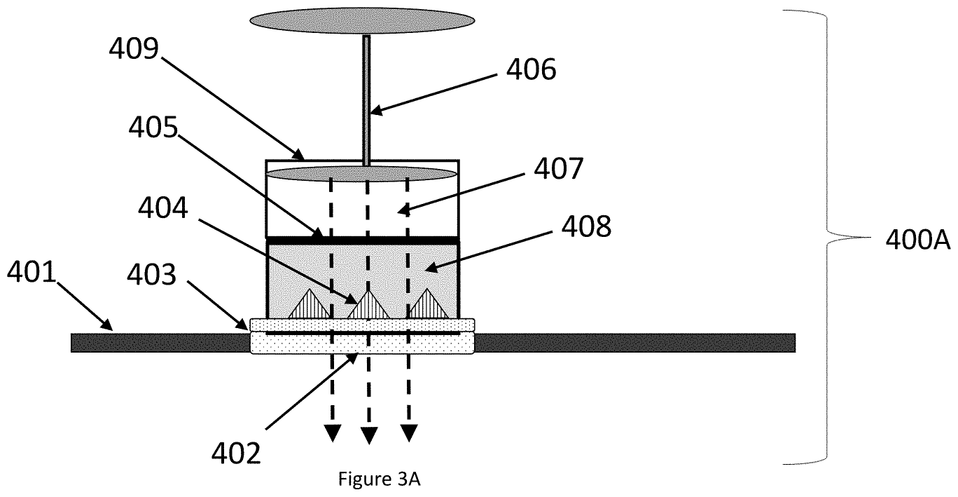

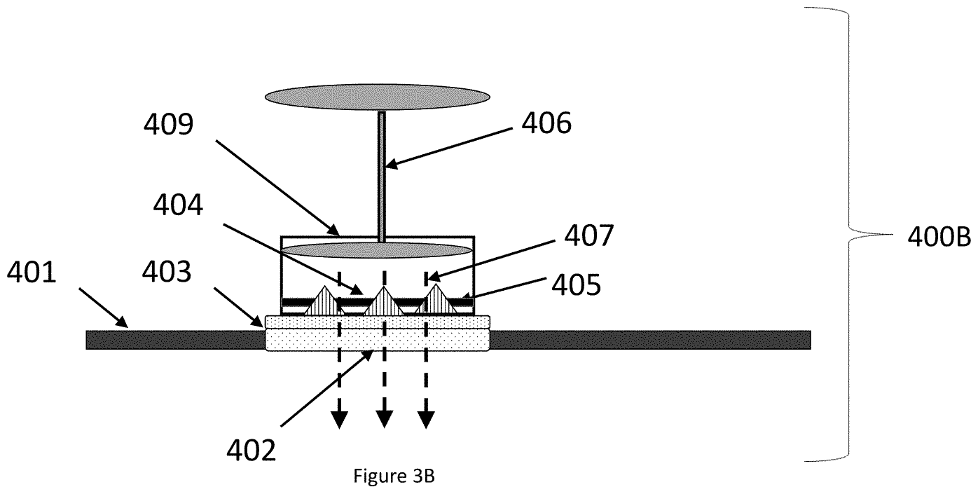

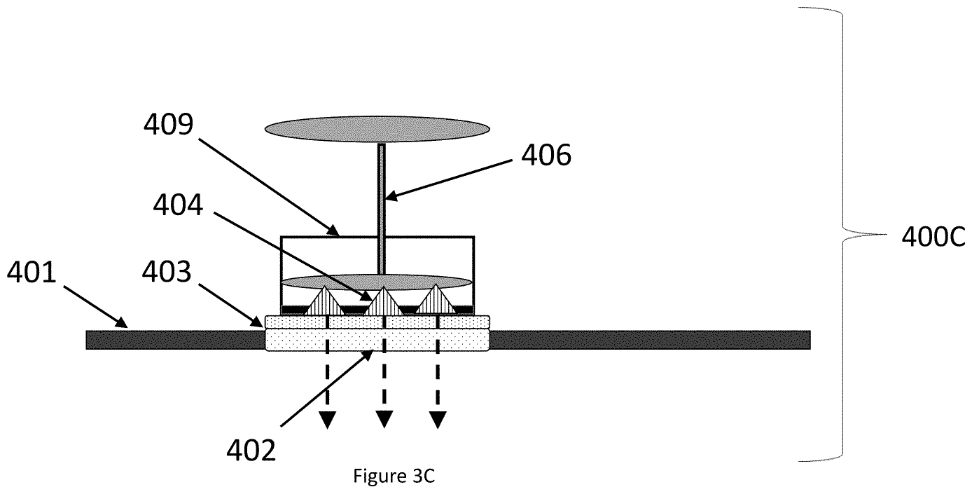

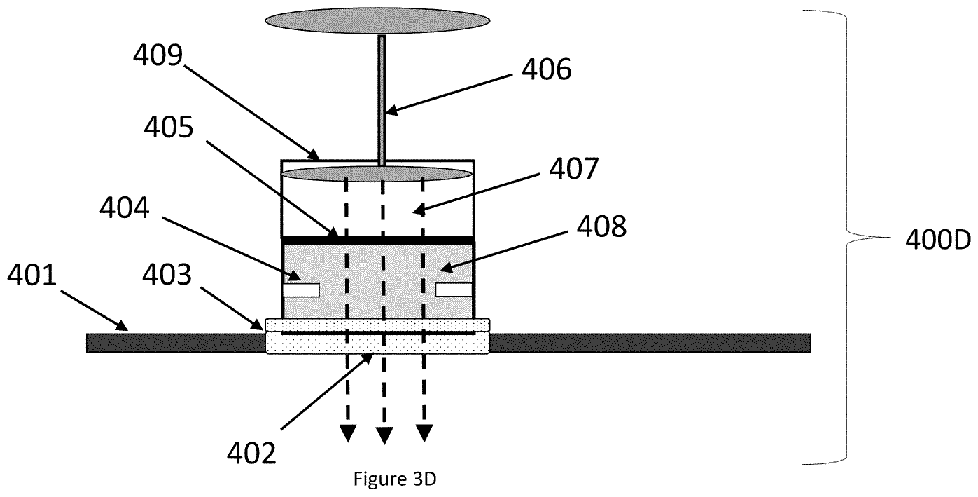

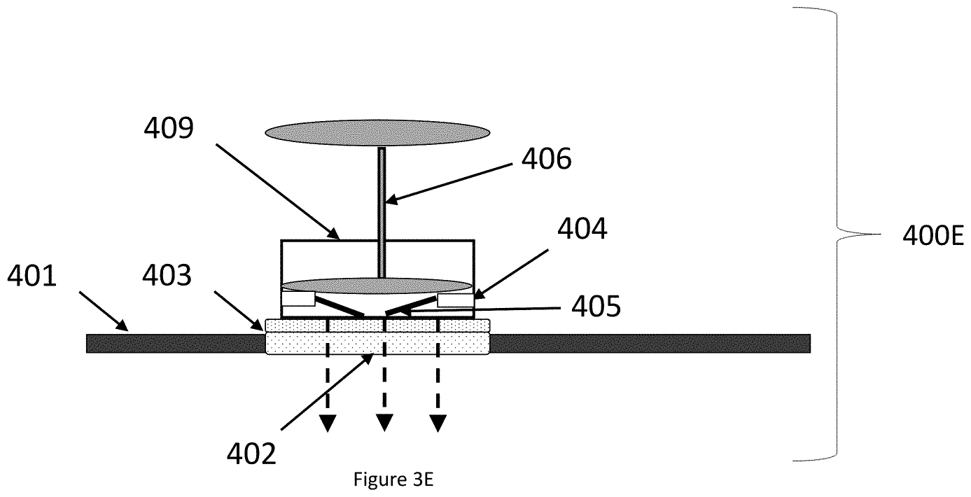

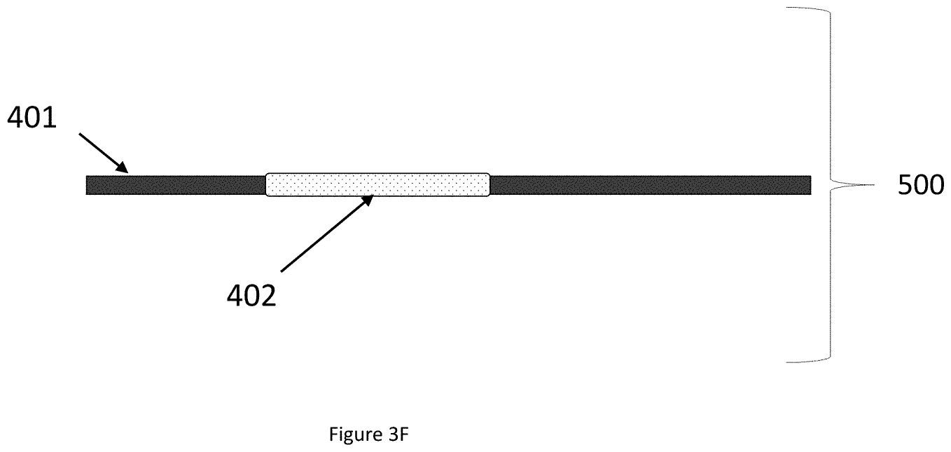

Reference is made to FIG. 3A showing a non-limiting configuration of the disclosed system 400A. In an exemplary configuration, a receptacle (also referred to as “vial” or “syringe”) 409 may have two compartments 407 and 408, separated by a barrier (e.g., membrane) 405. One compartment 407 may contain a buffer, the other 408 may contain fluid under test (e.g. a liquid sample such as urine).

Optionally, sample from compartment 408 may be pushed, for example, by piston 406 through first and second filters 402, 403, respectively.

Optionally, first filter 402 may be embedded into a microfluidic chip 401, while second filter 403 may be embedded into vial/syringe 409.

First filter 402 and second filter 403 may be configured similarly to the ITP Apparatus 200 as described hereinabove under “the apparatus”.

In another exemplary configuration, as shown in FIGS. 3B, 3C, and 3E, (systems 400B, 400C, and 400E, respectively) sample compartment 408 may be depleted, and, upon the depletion membrane 405 may be opened, disintegrated or punched by a barrier (e.g., membrane) opener 404, enabling flow of buffer through filters 402 and 403.

Optionally, membrane opener 404 may be disposed on membrane 403 allowing to punch membrane 405, as shown in FIGS. 3B and 3C.

In another exemplary configuration, as illustrated in FIGS. 3D and 3E, membrane opener 404 may be disposed on an internal wall of vial/syringe 409 e.g., in a hook-like shape, allowing to open or to break membrane 405 upon contacting membrane opener 404.

In an exemplary operation of systems 400B and 400E shown in FIGS. 3B and 3E, respectively, after the buffer is flown through the filters, the syringe 409 has no additional function and therefore may be removed (see illustration in system 500, FIG. 3F), and the analyte (e.g., bacteria) is allowed to remain on filter 402 and the test on chip 401 may run.

Optionally, the system as described herein further comprises a control unit.

Optionally, the control unit allows to control flow of the fluid sample through the filters.

Optionally, the control unit allows to control flow of the analyte through the microfluidic chip.

Optionally, the disclosed system further comprises a computer program product.

Optionally, the computer program product comprises a computer-readable storage medium. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer.

In some embodiments, the program code is excusable by a hardware processor.

In some embodiments, the hardware processor is a part of the control unit.

In some embodiments, there is further provided a read-out of the assay carried out in the disclosed system or device may be detected or measured using any suitable detection or measuring means known in the art. The detection means may vary depending on the nature of the read-out of the assay. In some embodiments, disclosed system also relates to an apparatus including the device in any embodiments thereof, and a detection means as described herein.

General:

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples

### EXAMPLES

Reference is now made to the following examples, which together with the above descriptions illustrate some embodiments of the invention in a non-limiting fashion.

Sample Preparation

Escherichia coli culture (JM109 strain, was grown in Luria-Bertani (LB) broth at 37° C. with vigorous shaking to an optical density of 0.3 at 600 nm (OD600), corresponding to approximately 1.8×10⁸ cfu/mL (1 OD=6×10⁸ cfu/mL), as measured by drop plate method. E. faecalis (Symbioflor 1 strain) was grown in MRS broth (1 OD=7×10⁸ cfu/mL).

Bacterial suspension was concentrated by centrifugation at 14,000×g for 2 minutes. Supernatant was removed and pellet was resuspended in 0.85% NaCl and washed by an additional centrifugation step to remove significant traces of interfering media components. Next, supernatant was removed, and pellet was resuspended in 0.85% NaCl and SYTO9 dye (Molecular probes, cat. # L-7002) was added to a final concentration of 10 μM. The suspension was mixed and incubated for 10 minutes at room temperature. To discard remaining free fluorophores, the suspension was centrifuged, supernatant was removed and the pellet was resuspended in TE buffer for a desired concentration. For detection of bacteria from urine using ITP, urine samples with spiked bacteria were incubated with 10 μM of SYTO9 dye for 5 minutes at RT prior to the filtration procedure which is described herein.

Seeding for Filtration Efficiency Experiments: Drop Plate Method:

Urine samples were vortexed and serial dilutions were performed for each sample, before and after filtration. Samples were expelled in four evenly spaced 25 μl drops onto the quadrant of the LB plates and drops were soaked into the media before turning plates over for incubation overnight at 37° C. When colonies had developed, the dilutions which contained 3-30 colonies per 25 μl drop were counted manually.

In exemplary procedures, Gram-positive and Gram-negative bacteria cultures were established, representative of bacteria types commonly encountered in UTI (Table 1), but which do not present a biohazard and are safe to work with in a lab.

For high accuracy, a correlation between the light absorbance of the cell culture sample at 600 nm illumination (OD600) was determined and the actual bacteria number, as obtained from standard plate counting.

TABLE 1
Bacteria cultures established during the feasibility period

| Bacteria | | | | |
|---|---|---|---|---|
| (species | Group | Biosafety | Growth | Culture |
| and strain) | type | level | atmosphere | media |
| E. coli | Gram-negative | 1 | Aerobic | LB with 100 |
| JM109 | | | | mg/mL |
| | | | | Ampicillin |
| E. faecalis | Gram-positive | 1 | Aerobic | MRS |
| Symbioflor 1 | | | | broth |

### Example 1

### Experimental Procedure

Bacteria in buffer (with centrifugation): Briefly, upon culturing, resuspended bacterial pellet was resuspended in 0.85% NaCl and added generic fluorescent SYTO9 dye to a final concentration of 10 μM. The suspension was incubated for 10 minutes at RT. To discard remaining free fluorophores, they were centrifuged, removing the supernatant and resuspended the pellet in TE buffer (10 mM tricine and 20 mM bistris) for a desired concentration. The purpose of these experiments was to test the ability to focus whole bacteria by ITP process using the LVF devices, without any aggregation or clogging phenomena.

Bacteria in urine (no centrifugation): bacteria were spiked into urine samples and incubated it with 10 μM of SYTO9 dye for 5 minutes at RT prior to the filtration procedure which is described in section 2.4. The purposes of these experiments were to demonstrate the ability to handle real urine samples without any centrifugation procedure and to show filtration system efficiency.

Bacteria in a clinical UTI sample (no centrifugation): serial dilutions of a single clinical UTI sample were performed and each serial dilution was incubated with 10 μM SYTO9 dye for 5 minutes at RT prior to the filtration procedure which is described herein. In total, five filtration procedures were performed, one for each serial dilution. The purpose of this experiment was to demonstrate the ability to handle clinical UTI samples.

### Example 2

### Evaluation of Filtration System Efficiency

To show the feasibility to a real sample, the filtration system efficiency was first evaluated in terms of bacteria losses, which may mainly occur due to possible binding to the filter membrane.

FIGS. 4A-B present experimental results demonstrating the disclosed filtration technique efficiently in terms of bacteria count. Initially, this technique was tested on urine samples spiked with bacteria (FIG. 4A) and later implemented it for a real urinary tract infection UTI urine sample (FIG. 4B).

It was demonstrated that the filtration system can be implemented for effective detection of UTI using urine samples.

Importantly, there are several ways to increase filtration yield. The simplest way is to process 10 times larger urine volume, and to elute the bacteria in the same volume as previous (allowing an additional order of magnitude concentration and thus compensating an initial loss). To show the filtration procedure is compatible with the ITP process, filtered urine samples in large volume focusing (LVF) devices were tested.

### Example 7

### Detection of Bacteria from Urine Samples

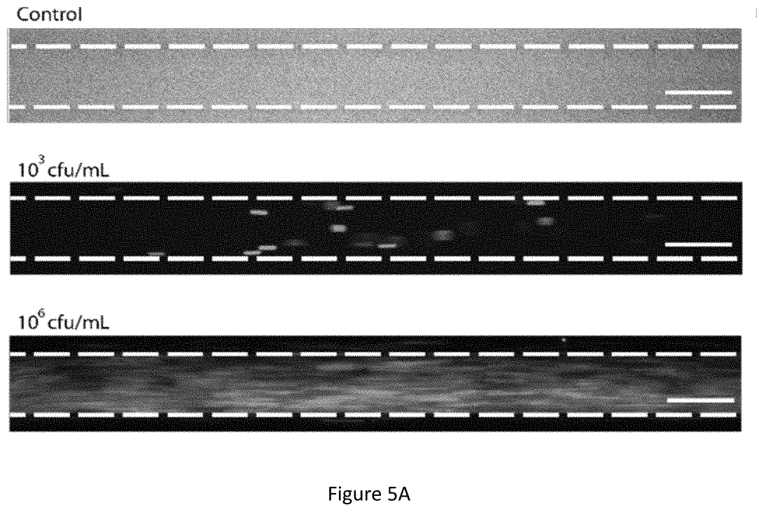

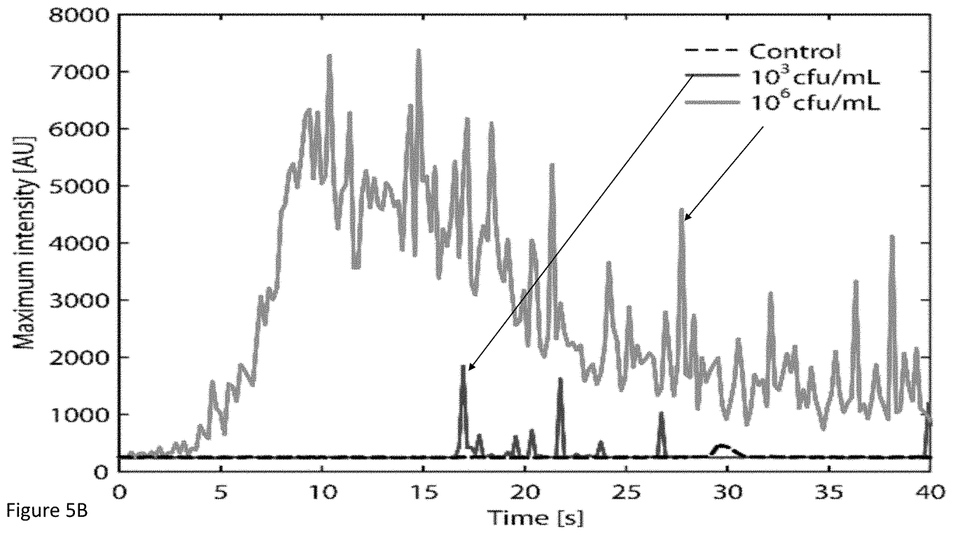

FIGS. 5A-B present bacterial focusing from urine samples spiked with bacteria following filtration procedure which is described herein. Results indicate that it is feasible to focus and detect bacteria from 10³ and 10⁶ cfu/mL filtered urine samples. Urine samples with spiked bacteria were incubated with 10 μM SYTO9 dye for 5 minutes at room temperature (RT) prior to the filtration procedure. All experiments were performed at 1100V, with the TE consisting of 10 mM tricine and 20 mM bistris; the LE consisting of 10 mM HCl, 20 mM pyridine and 1% (w/v) polyvinylpyrrolidone (PVP).

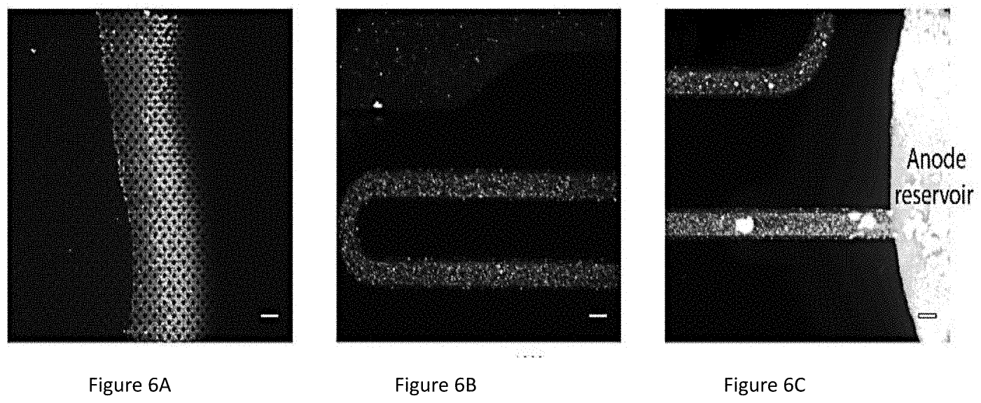

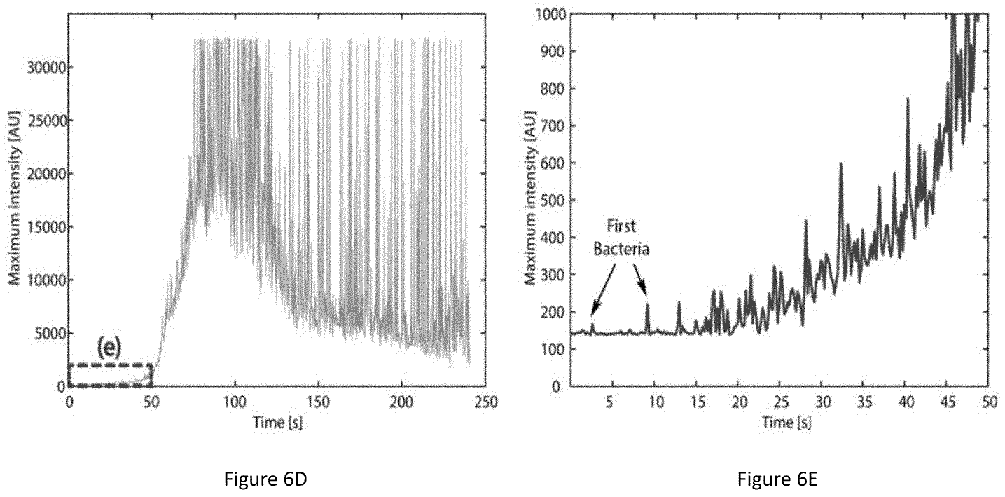

The results demonstrate that the maximum intensity signal obtained for 10³ cfu/mL was higher by an order of magnitude compared to the control sample. At high concentration (10⁶ cfu/mL) the signal was significantly elevated and a larger number of events (peaks in signal) was observed. Importantly, no aggregation or clogging of the channel was observed even for 10⁸ cfu/mL sample (FIGS. 6A-E).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An isotachophoresis (ITP) apparatus comprising:
(i) a first zone configured to contain a solution comprising a trailing electrolyte (TE);
(ii) a second zone configured to contain solution comprising a leading electrolyte (LE);
(iii) a flow channel connecting the first zone and the second zone; and
(iv) a first filter having a pore size sufficient to entrap an analyte, the first filter being integrated within the first zone, and in fluid communication with the flow channel, wherein said first filter has a pore size in the range of 0.1-1.0 μm;
wherein the flow channel is in a distinct direction with respect to the filtration flow.

2. The apparatus of claim 1, wherein the first filter and the flow channel are substantially in the same plane.

3. (canceled)

4. The apparatus of claim 1, further comprising a second filter having a pore size larger than the analyte.

5. The apparatus of claim 4, wherein said second filter is in fluid communication with said first filter.

6. The apparatus of claim 4, wherein said second filter is a detachable filter disposed atop the first filter.

7. The apparatus of claim 4, wherein the second filter is characterized by a pore size in the range of 0.5-10 μm.

8. The apparatus of claim 1, wherein the first zone and said second zone are configured to be operably connected to at least one anode and at least one cathode.

9. A system comprising:
(i) a microfluidic device comprising a flow channel;
(ii) a first filter having a pore size sufficient to entrap an analyte, and in fluid communication with the flow channel, the flow channel is in a distinct direction with respect to the filtration flow;
(iii) a receptacle divided by a membrane into a first compartment configured to contain a fluid sample and a second compartment configured to contain a buffer, and
(iv) a second filter having a pore size larger than the analyte,
the first compartment of the receptacle is configured to be placed in fluid communication with said flow channel through said filter,
the receptacle comprises a membrane-opening mechanism configured to allow flow of the buffer to the flow channel subsequent to flow of the fluid sample through the filter.

10. (canceled)

11. The system of claim 9, wherein said second filter is in fluid communication with said first filter.

12. The system of claim 9, wherein said second filter is a detachable filter disposed atop the first filter.

13. The system of claim 9, wherein the microfluidic device is an ITP apparatus comprising:
(i) a first zone configured to contain a solution comprising a TE;
(ii) a second zone configured to contain solution comprising an LE;
said flow channel connects the first zone and the second zone, and the first zone and said second zone are configured to be operably connected to at least one anode and at least one cathode.

14. An electrophoresis-sample preparation method comprising:
(i) a preliminary filtration step comprising filtering the fluid sample through a second filter having a pore size larger than the analyte;
(ii) a filtration step comprising filtering a fluid sample comprising an analyte through a first filter sufficient to entrap the analyte; and
(iii) a buffer exchange step comprising passing an electrophoresis buffer through the first filter;
thereby receiving an electrophoresis buffer comprising the analyte.

15. (canceled)

16. The method of claim 14, further comprising a step of labeling the analyte with a label detected under electrophoresis.

17. The method of claim 14, further comprising a step comprising: applying the electrophoresis buffer comprising the analyte to a flow channel, applying an electric potential along the flow channel, and detecting the analyte.

18. The method of claim 14, wherein said electrophoresis is ITP and said electrophoresis buffer is a solution comprising a TE.

19. The method of claim 14, wherein said fluid sample is urine.

20. The method of claim 14, wherein said analyte comprises a bacterium.

21. The method of claim 14, wherein the second filter is sufficient to remove white blood cells from the fluid sample.

22. The method of claim 14, for detection of urinary tract infections (UTI).
